(19) United States
(12) Patent Application Publication
(10) Pub. No.: US 2007/0197932 A1
(43) Pub. Date: Aug. 23, 2007

(54) Non-invasive methods for evaluating retinal affecting neurodegenerative diseases

(76) Inventors: Gilbert T. Feke, Stoneham, MA (US); Fatmire Berisha, Vienna (AT)

Correspondence Address: LAHIVE & COCKFIELD, LLP, ONE POST OFFICE SQUARE, BOSTON, MA, 02109-2127, US

(21) Appl. No.: 11/497,811

(22) Filed: Aug. 1, 2006

Related U.S. Application Data
(60) Provisional application No. 60/774,729, filed on Feb. 17, 2006.

(51) Int. Cl. A61B 13/00; A61B 5/00; A61B 8/00

(52) U.S. Cl. 600/558; 600/300; 600/455

(57) ABSTRACT

The present invention is directed to methods for detecting and evaluating retinal affecting neurodegenerative diseases. A plurality of selected retinal parameters are measured generating an eyeprint signature for a subject. The eyeprint signature can be used to evaluate whether the subject is suffering from a retinal affecting neurodegenerative disease, to monitoring the progression of the neurodegenerative disease, as well as to monitor the effectiveness of a treatment for the neurodegenerative disease.

### RELATED APPLICATIONS

This application claims priority to U.S. Provisional Patent Application 60/774,729, filed on Feb. 17, 2006. The entire contents of this patent application are hereby expressly incorporated herein by reference including, without limitation, the specification, claims, and abstract, as well as any figures, tables, or drawings thereof.

### FIELD OF THE INVENTION

This invention relates to methods for detecting and evaluating retinal affecting neurodegenerative diseases. More specifically, this invention provides methods for determining whether a subject is suffering from a neurodegenerative disorder by generating an eyeprint signature for a subject using selected retinal parameters, e.g., retinal nerve fiber layer thickness, retinal blood vessel diameter, retinal blood flow.

### BACKGROUND

Apoptotic nerve cell death is implicated in the pathogenesis of several devastating neurodegenerative conditions, including Alzheimer's disease, Parkinson's disease, and glaucoma. In many subjects suffering from a neurodegenerative condition, there is an impairment in visual function as well. Jackson, G. R., et al. Neurol Clin N Am 21(2003)709-728. Although many of the visual problems associated with such neurodegenerative conditions are due to cortical dysfunction, and eye movement abnormalities, there is evidence that optic nerve dysfunction, due to retinal ganglion cell loss may also occur. Sadun, A. A., and Bassi, C. J., Ophthalmology (1990) 97:9-17.

There have been numerous studies examining the effects of neurodegenerative diseases, e.g., Alzheimer's disease, Parkinson's disease, etc., on the retinal nerve fiber layer. In one of the first studies to examine the optic nerves and retinas of patients who had a neurodegenerative disease, i.e., Alzheimer's disease, widespread axonal degeneration in the optic nerve was found. Additionally, histological studies on the retinas of three of four of the patients studied found a reduction in the number of ganglion cells and in the thickness of the nerve fiber layer. Hinton, D. R., et al., N Engl J Med (1986); 315:485-7.

Blanks et al. extended this study to include the light microscopic and ultrastructural characteristics of retinal ganglion cell degeneration in a large number of patients with Alzheimer's disease. Blanks et al., Brain Res (1989); 501:364-72. Of the 16 Alzheimer's patients studied, 4 retinas were classified as having severe degeneration in the ganglion cell layer, 4 as moderate, 6 as mild, and 2 cases showed no apparent retinal pathology. Of the most severely affected retinas, there was a marked dropout of retinal ganglion cells associated with atrophy of the nerve fiber layer.

Numerous non-histological, in vivo methods have also been employed to study the retinal effects of neurodegenerative diseases. Tsai et al (Arch Ophthalmol (1991);109:199-203) and Hedges et al (Acta Ophthalmol Scand(1996); 74:271-75) used retinal photography to examine the retinal nerve fiber layer and optic nerve head in Alzheimer's patients. Their findings added to the histological evidence that ganglion cell degeneration occurs in Alzheimer's patients.

Although the above studies, both histological and in vivo, shed light on the effects neurodegenerative diseases may have on the retina, there are many drawbacks to the employed methods. Among the histological studies, differences in cell identification criteria, and difficulty in accurately making optic nerve fiber counts, for example, can lead to discrepancies in the results. Photographic studies require a more subjective evaluation by the observer.

Kergoat et al., using scanning laser polarimetry, found no difference in the retinal nerve fiber layer thickness between patients suffering from dementia of the Alzheimer type and controls. Kergoat et al., Acta Ophthalmol Scand (2001); 79:187-191. Kergoat et al. concluded that the retinal ganglion cells, and their axons, are not altered in patients with dementia of the Alzheimer type. However, while Kergoat et al. found no abnormalities in eyes with Alzheimer's disease, other studies have reported abnormalities. Parisi V. et al., Clin Neurophysiol (2001) 112:1860-1867; Iseri P. K., et al., J Neuroophthalmol (2006) 26:18-24.

It has also been suggested that a retinal circulatory abnormality is likely to accompany or precede the morphological changes found in the retina of patients suffering from Alzheimer's disease. This suggestion is based on the similarity in the retinal degeneration seen in Alzheimer's disease patients, and glaucoma patients, as well as the fact that retinal and optic nerve head blood flow is abnormally decreased in patients with glaucoma. Flammer J., et al., J Glaucoma 1999; 8:212-219.

### SUMMARY

The present invention provides objective, non-invasive, in vivo methods for diagnosing and/or monitoring the progression-of a neurodegenerative disease. Selected retinal parameters are used to generate an eyeprint signature for a subject. The eyeprint signature can be used to objectively determine the presence and/or progression of a neurodegenerative disease, as well as to monitor the therapeutic efficacy of a treatment.

Accordingly, in one aspect, the present invention is directed to a method for diagnosing a neurodegenerative disease in a subject. The method includes generating an eyeprint signature for a subject based on measurements of a plurality of selected retinal parameters. The method further includes diagnosing whether the subject has the neurodegenerative disease based on a comparison between the eyeprint signature of the subject and a standard eyeprint signature for the neurodegenerative disease.

In some embodiments, the selected retinal parameters are at least two parameters selected from the group consisting of the thickness of a retinal nerve fiber layer, the diameter of a retinal blood vessel, and a retinal blood flow rate. In some embodiments, the thickness of the retinal nerve fiber layer is measured using an optical coherence tomography machine. In other embodiments, the thickness of the superior, temporal, inferior, and/or nasal quadrants of the retinal nerve fiber layer are measured.

In some embodiments, the diameter of the retinal blood vessel is measured using a laser Doppler blood flowmeter. In some embodiments, the retinal blood flow rate is measured using a laser Doppler blood flowmeter.

In yet another embodiment, the neurodegenerative disease is a neurodegenerative disease of the eye. In still yet another embodiment, the neurodegenerative disease is selected from the group consisting of inflammatory optic neuropathy, macular degeneration, glaucoma, retinitis pigmentosa, and diabetic retinopathy. In other embodiments, the neurodegenerative disease is selected from the group consisting of Alzheimer's disease, Parkinson's disease, multiple sclerosis, amyotrophic lateral sclerosis, and Huntington's disease.

In other aspects the present invention is directed to a method for diagnosing Alzheimer's disease in a subject. The method includes generating an eyeprint signature for a subject based on measurements of a plurality of selected retinal parameters. The method further includes diagnosing whether the subject has Alzheimer's disease based on a comparison between the eyeprint signature of the subject and a standard eyeprint signature for Alzheimer's disease.

In some embodiments, the selected retinal parameters are at least two parameters selected from the group consisting of the thickness of a retinal nerve fiber layer, the diameter of a retinal blood vessel, and a retinal blood flow rate. In other embodiments, the thickness of the retinal nerve fiber layer is measured using an optical coherence tomography machine. In other embodiments, the thickness of the superior, temporal, inferior, and/or nasal quadrants of the retinal nerve fiber layer are measured.

In still other embodiments, the diameter of the retinal blood vessel is measured using a laser Doppler blood flowmeter. In other embodiments, the retinal blood flow rate is measured using a laser Doppler blood flowmeter.

In some embodiments, the standard eyeprint signature for Alzheimer's disease comprises a decreased thickness of the retinal nerve fiber layer, a decreased diameter of the retinal blood vessel, and a decreased blood flow rate based on a comparison to a control eyeprint signature. In some embodiments, the superior quadrant of the retinal nerve fiber layer is measured. It other embodiments, the standard eyeprint signature for Alzheimer's disease comprises a superior quadrant retinal nerve fiber layer thickness in the range from about 70 microns to about 105 microns, a retinal blood vessel diameter in the range from about 122 microns to about 142 microns, and a retinal blood flow in the range from about 8 μL/min to about 18 μL/min.

In other aspects, the present invention provides a method for diagnosing Parkinson's disease in a subject. The method includes generating an eyeprint signature for a subject based on measurements of a plurality of selected retinal parameters. The method further includes diagnosing whether the subject has Parkinson's disease based on a comparison between the eyeprint signature of the subject and a standard eyeprint signature for Parkinson's disease.

In other embodiments, the selected retinal parameters are at least two parameters selected from the group consisting of the thickness of a retinal nerve fiber layer, the diameter of a retinal blood vessel, and a retinal blood flow rate. In some embodiments, the thickness of the retinal nerve fiber layer is measured using an optical coherence tomography machine. In other embodiments, the thickness of the superior, temporal, inferior, and/or nasal quadrants of the retinal nerve fiber layer are measured. In some embodiments, the diameter of the retinal blood vessel is measured using a laser Doppler blood flowmeter. In other embodiments, the retinal blood flow rate is measured using a laser Doppler blood flowmeter.

In some aspects, the present invention provides a method for diagnosing glaucoma in a subject. The method includes generating an eyeprint signature for a subject based on measurements of a plurality of selected retinal parameters. The method further includes diagnosing whether the subject has glaucoma based on a comparison between the eyeprint signature of the subject and a standard eyeprint signature for glaucoma.

In some embodiments, the selected retinal parameters are at least two parameters selected from the group consisting of the thickness of a retinal nerve fiber layer, the diameter of a retinal blood vessel, and a retinal blood flow rate. In other embodiments, the thickness of the retinal nerve fiber layer is measured using an optical coherence tomography machine. In some embodiments, the thickness of the superior, temporal, inferior, and/or nasal quadrants of the retinal nerve fiber layer are measured. In some embodiments, the diameter of the retinal blood vessel is measured using a laser Doppler blood flowmeter. In other embodiments, the retinal blood flow rate is measured using a laser Doppler blood flowmeter.

In some embodiments, the standard eyeprint signature for glaucoma comprises a decreased thickness of the retinal nerve fiber layer, a decreased diameter of the retinal blood vessel, and a decreased blood flow rate based on a comparison to a control eyeprint signature. In some embodiments, the inferior and temporal quadrants of the retinal nerve fiber layer are measured.

In some embodiments, a standard eyeprint signature for glaucoma comprises an inferior quadrant retinal nerve fiber layer thickness in the range from about 74 microns to about 99 microns, a retinal blood vessel diameter in the range from about 88 microns to about 123 microns, and a retinal blood flow in the range from about 5 μL/min to about 13 μL/min.

In other aspects, the present invention is directed to a method for diagnosing inflammatory optic neuropathy in a subject. The method includes generating an eyeprint signature for a subject based on measurements of a plurality of selected retinal parameters. The method further includes diagnosing whether the subject has inflammatory optic neuropathy based on a comparison between the eyeprint signature of the subject and a standard eyeprint signature for inflammatory optic neuropathy.

In some embodiments, the selected retinal parameters are at least two parameters selected from the group consisting of the thickness of a retinal nerve fiber layer, the diameter of a retinal blood vessel, and a retinal blood flow rate. In other embodiments, the thickness of the retinal nerve fiber layer is measured using an optical coherence tomography machine. In other embodiments, the thickness of the superior, temporal, inferior, and/or nasal quadrants of the retinal nerve fiber layer are measured.

In some embodiments, the diameter of the retinal blood vessel is measured using a laser Doppler blood flowmeter. In other embodiments, the retinal blood flow rate is measured using a laser Doppler blood flowmeter.

In some embodiments, the standard eyeprint signature for inflammatory optic neuropathy comprises a decreased thickness of the retinal nerve fiber layer, a normal diameter of the retinal blood vessel, and a normal blood flow rate based on a comparison to a control eye print signature. In some embodiments, the superior and nasal quadrants of the retinal nerve fiber layer are measured. In other embodiments, a standard eyeprint signature for inflammatory optic neuropathy comprises a superior quadrant retinal nerve fiber layer thickness in the range from about 70 microns to about 100 microns, an inferior quadrant retinal nerve fiber layer thickness in the range from about 101 to about 130, a retinal blood vessel diameter in the range from about 133 microns to about 153 microns, and a retinal blood flow in the range from about 12 μL/min to about 23 μL/min.

In some aspects, the present invention is directed to a method for monitoring a therapeutic treatment for a neurodegenerative disease. The method includes generating a monitoring eyeprint signature for a subject based on a plurality of selected retinal parameters. The method further includes comparing the monitoring eyeprint signature to a threshold eyeprint signature for the subject, and determining the effectiveness of the therapeutic treatment.

In some embodiments, the selected retinal parameters are at least two parameters selected from the group consisting of the thickness of a retinal nerve fiber layer, the diameter of a retinal blood vessel, and a retinal blood flow rate. In some embodiments, the thickness of the retinal nerve fiber layer is measured using an optical coherence tomography machine. In other embodiments, the thickness of the superior, temporal, inferior, and/or nasal quadrants of the retinal nerve fiber layer are measured. In other embodiments, the diameter of the retinal blood vessel is measured using a laser Doppler blood flowmeter. In some embodiments, the retinal blood flow rate is measured using a laser Doppler blood flowmeter.

In some embodiments, the neurodegenerative disease is a neurodegenerative disease of the eye. In some embodiments, the neurodegenerative disease is selected from the group consisting of optic neuropathy, macular degeneration, glaucoma, retinitis pigmentosa, and diabetic retinopathy. In other embodiments, the neurodegenerative disease is selected from the group consisting of Alzheimer's disease, Parkinson's disease, multiple sclerosis, amyotrophic lateral sclerosis, and Huntington's disease.

In some embodiments, the neurodegenerative disease is Alzheimer's disease. In some embodiments, the selected retinal parameters comprising the monitoring eyeprint are at least two parameters selected from the group consisting of the thickness of a retinal nerve fiber layer, the diameter of a retinal blood vessel, and the retinal blood flow rate. In some embodiments, the thickness of the retinal nerve fiber layer is measured using an optical coherence tomography machine. In other embodiments, the thickness of the superior quadrant of the retinal nerve fiber layer is measured. In other embodiments, the diameter of the retinal blood vessel is measured using a laser Doppler blood flowmeter. In some embodiments, the retinal blood flow rate is measured using a laser Doppler blood flowmeter.

In some embodiments, a decrease in the values of the selected retinal parameters comprising the monitoring eyeprint based on a comparison to the threshold eyeprint indicates an ineffective treatment.

In some embodiments, the neurodegenerative disease is Parkinson's disease. In other embodiments, the selected retinal parameters comprising the monitoring eyeprint are at least the retinal nerve fiber layer thickness, retinal blood vessel diameter, and retinal blood flow.

In some embodiments, the thickness of the retinal nerve fiber layer is measured using an optical coherence tomography machine. In other embodiments, the thickness of the superior quadrant of the retinal nerve fiber layer is measured. In some embodiments, the diameter of the retinal blood vessel is measured using a laser Doppler blood flowmeter. In other embodiments, the retinal blood flow rate is measured using a laser Doppler blood flowmeter.

In some embodiments, the neurodegenerative disease is glaucoma. In other embodiments, the selected retinal parameters comprising the monitoring eyeprint are at least the retinal nerve fiber layer thickness, the retinal blood vessel diameter, and the retinal blood flow. In some embodiments, the thickness of the retinal nerve fiber layer is measured using an optical coherence tomography machine. In other embodiments, the diameter of the retinal blood vessel is measured using a laser Doppler blood flowmeter. In some embodiments, the retinal blood flow rate is measured using a laser Doppler blood flowmeter.

In other embodiments, the thickness of the inferior and temporal quadrants of the retinal nerve fiber layer are measured. In some embodiments, a decrease in the values of the selected retinal parameters comprising the monitoring eyeprint based on a comparison to the threshold eyeprint indicates an ineffective treatment. In some embodiments, the neurodegenerative disease is inflammatory optic neuropathy. In other embodiments, the selected retinal parameters comprising the monitoring eyeprint are at least the retinal nerve fiber layer thickness, retinal blood vessel diameter, and retinal blood flow. In other embodiments, the thickness of the retinal nerve fiber layer is measured using an optical coherence tomography machine. In some embodiments, the thickness of the superior and nasal quadrants of the retinal nerve fiber layer are measured. In other embodiments, the diameter of the retinal blood vessel is measured using a laser Doppler blood flowmeter. In some embodiments, the retinal blood flow rate is measured using a laser Doppler blood flowmeter.

In some embodiments, a decrease in the thickness of the retinal nerve fiber layer and an increase in the blood flow rate measurements comprising the monitoring eyeprint based on a comparison to the threshold eyeprint indicates an ineffective treatment.

In another aspect, the present invention provides a method for monitoring the progression of a neurodegenerative disease. The method includes generating a monitoring eyeprint signature for a subject based on a plurality of selected retinal parameters. The method further includes comparing the monitoring eyeprint signature to a threshold eyeprint signature for the subject, and determining the progression of the neurodegenerative disease.

In some embodiments, the selected retinal parameters are at least two parameters selected from the group consisting of the thickness of a retinal nerve fiber layer, the diameter of a retinal blood vessel, and a retinal blood flow rate. In other embodiments, the thickness of the retinal nerve fiber layer is measured using an optical coherence tomography machine. In some embodiments, the thickness of the superior, temporal, inferior, and/or nasal quadrants of the retinal nerve fiber layer are measured. In other embodiments, the diameter of the retinal blood vessel is measured using a laser Doppler blood flowmeter. In other embodiments, the retinal blood flow rate is measured using a laser Doppler blood flowmeter.

In some embodiments, the neurodegenerative disease is a neurodegenerative disease of the eye. In some embodiments, the neurodegenerative disease is selected from the group consisting of inflammatory optic neuropathy, macular degeneration, glaucoma, retinitis pigmentosa, and diabetic retinopathy. In other embodiments, the neurodegenerative disease is selected from the group consisting of Alzheimer's disease, Parkinson's disease, multiple sclerosis, amyotrophic lateral sclerosis, and Huntington's disease.

In some embodiments, the neurodegenerative disease is Alzheimer's disease. In some embodiments, the selected retinal parameters comprising the monitoring eyeprint are at least the retinal nerve fiber layer thickness, the retinal blood vessel diameter, and the retinal blood flow. In other embodiments, the thickness of the retinal nerve fiber layer is measured using an optical coherence tomography machine. In yet other embodiments, the thickness of the superior quadrant of the retinal nerve fiber layer is measured.

In some embodiments, the diameter of the retinal blood vessel is measured using a laser Doppler blood flowmeter. In other embodiments, the retinal blood flow rate is measured using a laser Doppler blood flowmeter. In some embodiments, a decrease in the values of the selected retinal parameters comprising the monitoring eyeprint based on a comparison to the threshold eyeprint indicates the progression of Alzheimer's disease.

In some embodiments, the neurodegenerative disease is Parkinson's disease. In some embodiments, the selected retinal parameters comprising the monitoring eyeprint are at least the retinal nerve fiber layer thickness, retinal blood vessel diameter, and retinal blood flow. In other embodiments, the thickness of the retinal nerve fiber layer is measured using an optical coherence tomography machine. In other embodiments, the thickness of the superior quadrant of the retinal nerve fiber layer is measured. In some embodiments, the diameter of the retinal blood vessel is measured using a laser Doppler blood flowmeter. In other embodiments, the retinal blood flow rate is measured using a laser Doppler blood flowmeter.

In some embodiments, the neurodegenerative disease is glaucoma. In some embodiments, the selected retinal parameters comprising the monitoring eyeprint are at least the retinal nerve fiber layer thickness, the retinal blood vessel diameter, and the retinal blood flow. In other embodiments, the thickness of the retinal nerve fiber layer is measured using an optical coherence tomography machine. In other embodiments, the diameter of the retinal blood vessel is measured using a laser Doppler blood flowmeter. In other embodiments, the retinal blood flow rate is measured using a laser Doppler blood flowmeter. In some embodiments, the thickness of the inferior and temporal quadrants of the retinal nerve fiber layer are measured.

In some embodiments, a decrease in the values of the selected retinal parameters comprising the monitoring eyeprint based on a comparison to the threshold eyeprint indicates an ineffective treatment.

In other embodiments, the neurodegenerative disease is inflammatory optic neuropathy. In some embodiments, the selected retinal parameters comprising the monitoring eyeprint are at least the retinal nerve fiber layer thickness, retinal blood vessel diameter, and retinal blood flow. In other embodiments, the thickness of the retinal nerve fiber layer is measured using an optical coherence tomography machine. In other embodiments, the thickness of the superior and nasal quadrants of the retinal nerve fiber layer are measured. In some embodiments, the diameter of the retinal blood vessel is measured using a laser Doppler blood flowmeter. In other embodiments, the retinal blood flow rate is measured using a laser Doppler blood flowmeter.

In some embodiments, a decrease in the thickness of the retinal nerve fiber layer measurement comprising the monitoring eyeprint based on a comparison to the threshold eyeprint indicates an ineffective treatment.

In another aspect, the present invention is directed to a method of screening a subject for a neurodegenerative disease. The method includes comparing an eyeprint signature for a subject to a standard eyeprint signature for the neurodegenerative disease wherein a correlation between the eyeprint signature for the subject and the standard eyeprint signature for the neurodegenerative disease indicates the presence of a neurodegenerative disease such that the subject is screened.

In some embodiments, the neurodegenerative disease is a neurodegenerative disease of the eye. In some embodiments, the neurodegenerative disease is selected from the group consisting of inflammatory optic neuropathy, macular degeneration, glaucoma, retinitis pigmentosa, and diabetic retinopathy. In other embodiments, the neurodegenerative disease is selected from the group consisting of Alzheimer's disease, Parkinson's disease, multiple sclerosis, amyotrophic lateral sclerosis, and Huntington's disease.

In some embodiments, the neurodegenerative disease is Alzheimer's disease. In some embodiments, the standard eyeprint signature for Alzheimer's disease comprises a superior quadrant retinal nerve fiber layer thickness in the range from about 70 microns to about 105 microns, a retinal blood vessel diameter in the range from about 122 microns to about 142 microns, and a retinal blood flow in the range from about 8 μL/min to about 18 μL/min.

In some embodiments, the neurodegenerative disease is Parkinson's disease. In other embodiments, the neurodegenerative disease is glaucoma. In some embodiments, the standard eyeprint signature for glaucoma comprises an inferior quadrant retinal nerve fiber layer thickness in the range from about 74 microns to about 99 microns, a retinal blood vessel diameter in the range from about 88 microns to about 123 microns, and a retinal blood flow in the range from about 5 μL/min to about 13 μL/min.

In other embodiments, the neurodegenerative disease is inflammatory optic neuropathy. In some embodiments, the standard eyeprint signature for inflammatory optic neuropathy comprises an superior retinal nerve fiber layer thickness in the range from about 70 microns to about 100 microns, an inferior quadrant retinal nerve fiber layer thickness in the range from about 101 microns to about 130 microns, a retinal blood vessel diameter in the range from about 133 microns to about 153 microns, and a retinal blood flow in the range from about 12 μL/min to about 23 μL/min.

### DETAILED DESCRIPTION

The present invention relates to methods for diagnosing and/or monitoring the progression of neurodegenerative diseases that affect the retina. Methods for evaluating therapeutic treatments used to treat the neurodegenerative disease are also provided.

### Retinal Structure

The retina is a multi-layered, light sensitive membrane that lines the back of the eye, and is connected to the brain by the optic nerve. The optic nerve transmits the electrical impulses received from the retina to the brain.

All vertebrate retinas are composed of three layers of nerve cell bodies and two layers of synapses. The three layers of nerve cell bodies are the outer nuclear layer, the inner nuclear layer and the ganglion cell layer. The outer nuclear layer contains cell bodies of the rods and cones. The inner nuclear layer contains cell bodies of the bipolar, horizontal and amacrine cells. The ganglion cell layer contains cell bodies of ganglion cells and displaced amacrine cells. Dividing these nerve cell layers are two neuropils where synaptic contacts occur.

The innermost limit of the retina is the inner limiting membrane (ILM). The ILM is formed by the fused feet of the Muller cells, the retina's glial element. The ILM serves to seal off the retina's neural element from the vitreous body.

The nerve fiber layer (NFL) is where axons of the ganglion cells are bundled together to form the origins of the optic nerve. These fibers are derived from cell bodies of ganglion cells, which are located in the ganglion cell layer (GCL). The ganglion cells are the final neuron in the chain that sends information to the visual nucleus.

The ganglion cells take their input via synapses in the inner plexiform layer (IPL). Ganglion cell dendrites join there with axons from bipolar cells. The bipolar cells are neurons whose cell bodies comprise the inner nuclear layer (INL). Integrator neurons (the horizontal and amacrine cells) also have their bodies in the inner nuclear layer.

The outer plexiform layer (OPL) contains the dendrites of the bipolar cells, and their synapses with the axons of rod and cone cells. The somata of the rods and cones reside in the outer nuclear layer (ONL).

The actual light-sensitive parts of the rods and cones are also sealed off from the rest of the system via the outer limiting membrane (OLM). The OLM is not a membrane, but rather a region of occluding junctions between Muller cells and the rod and cone cells.

Finally, the layer of rods and cones (R&CL) are the actual light sensitive elements. The pigment cell layer (PCL) is strictly speaking not part of the retina proper. The PCL participates in phagocytosis of the used-up rod and cone material (which is replaced) and in the cycle by which visual pigments are formed.

Light travels through the thickness of the retina before striking and activating the rods and cones. Subsequently the absorbtion of photons by the visual pigment of the photoreceptors is translated into first a biochemical message and then an electrical message that can stimulate all the succeeding neurons of the retina. The retinal message concerning the photic input and some preliminary organization of the visual image into several forms of sensation are transmitted to the brain from the spiking discharge pattern of the ganglion cells.

As mentioned above, the nerve fiber layer is the layer of the retina at which the optic nerve originates. The optic nerve contains the ganglion cell axons running to the brain and, additionally, incoming blood vessels that open into the retina to vascularize the retinal layers and neurons. Without wishing to be bound by any particular theory, it is hypothesized that because the retina is connected to the brain through the optic nerve, changes that affect the brain should therefore affect the retina.

### Neurodegenerative Diseases

In one embodiment, the present invention relates to methods for detecting or diagnosing neurodegenerative diseases or disorders. As used herein, the terms “neurodegenerative disease” and “neurodegenerative disorder” include diseases the pathology of which involves neuronal degeneration and/or dysfunction. The term neurodegenerative disease includes Alzheimer's disease (AD), Parkinson's disease, epilepsy, multiple sclerosis, amyotrophic lateral sclerosis (ALS), Huntington's disease, macular degeneration, glaucoma, inflammatory optic neuropathy, retinitis pigmentosa, and diabetic retinopathy.

In one embodiment, the present invention provides methods for diagnosing and/or monitoring the progression of Alzheimer's disease, as well as methods for determining the effectiveness of a therapeutic treatment for Alzheimer's disease. Alzheimer's disease is art recognized and is the most common neurodegenerative disorder of aging. Alzheimer's disease is characterized by progressive dementia in middle and late life. The abnormal accumulation of amyloid plaques in the vicinity of degenerating neurons and reactive astrocytes is a pathological characteristic of Alzheimer's disease.

A clinical diagnosis of Alzheimer's disease is typically based on the following criteria: (1) dementia established by clinical examination and documented by the Mini-Mental State Examination; (2) deficits in two or more areas of cognition; (3) no disturbance of consciousness; (4) onset between the ages of 40 and 90; and (5) absence of a systemic disorder or other neurological disease that could account for the progressive deficits in memory or cognition. Katz, B., et al., Survey Ophthamol, 34:31-43, 1989. Many patients with Alzheimer's also experience visual sensory problems including, reduced acuity, impaired spatial contrast sensitivity, achromatopsia, impaired stereopsis, deficits in perceiving shape from motion, and slowed visual processing speed.

In another embodiment, the present invention provides methods for diagnosing and/or monitoring the progression of Parkinson's disease, as well as methods for determining the effectiveness of a therapeutic treatment for Parkinson's disease. Parkinson's disease is art recognized and is a disorder in which neurons in the substantia nigra region of the brain die or become impaired. Normally, these cells produce a vital chemical known as dopamine. Dopamine allows smooth, coordinated function of the body's muscles and movement. When approximately 80% of the dopamine-producing cells are damaged, the symptoms of Parkinson disease appear. The symptoms of Parkinson's include, but are not limited to, tremors, rigidity, and difficulty and slowness of movement.

Making an accurate diagnosis in the early stages of Parkinson's disease can be difficult, and may require observation of the patient for some time until it is apparent that the tremor is consistently present and is joined by one or more of the other symptoms. Currently, there are no specific tests for diagnosing Parkinson's disease, although there are several methods for evaluating its presence, including the following: neurological examination (including evaluation of symptoms and their severity); trial test of drugs—when symptoms are significant, a trial test of drugs (primarily levodopa [L-dopa]) may be used to further diagnose the presence of PD. If a patient fails to benefit from levodopa, a diagnosis of Parkinson's disease may be questionable; CT scans; and magnetic resonance imaging (MRI).

In yet another embodiment, the present invention provides methods for diagnosing and/or monitoring the progression of inflammatory optic neuropathy, as well as methods for determining the effectiveness of a therapeutic treatment for inflammatory optic neuropathy. There are three classes of inflammatory optic neuropathies: (1) demyelinating disease, e.g., multiple sclerosis; (2) infectious disease, e.g., meningitis or lyme disease; and (3) immune mediated disorders, e.g., sarcoidosis. Inflammatory optic neuropathies can be acute or chronic and are typically characterized by any of the following: unilateral vision loss progressing over hours or days; sequential involvement of the opposite eye; visual field defects, especially central visual field loss; diminished color perception and difficulty seeing in dim light; pain in or around the eye; and visual phenomena.

In a further embodiment, the present invention provides methods for diagnosing and/or monitoring the progression of glaucoma, as well as methods for determining the effectiveness of a therapeutic treatment for glaucoma. Glaucoma is a disease characterized by damage to the optic nerve that is often, but not always, associated with high intraocular pressure. Traditional methods of diagnosing glaucoma include, measuring the pressure in the eye using a tonometer, visual field testing, and examining the optic nerve using an ophthalmoscope.

### Methods of Generating an Eyeprint Signature

The present invention provides methods for diagnosing and/or monitoring the progression of a neurodegenerative disease. The present invention also provides methods for determining the effectiveness of a therapeutic treatment of the neurodegenerative disease.

In one embodiment, an eyeprint signature for a subject is generated based on measurements of a plurality of selected retinal parameters. The term “eyeprint signature” as used herein, refers to a plurality of retinal parameters that are sufficient to allow one to diagnosis the presence or absence of a neurodegenerative disease. For example, a plurality of retinal parameters are measured in a subject or group of subjects. The retinal parameters chosen indicate the current structural and functional characteristics of the retina. The measurements of these parameters are compiled and form the basis for the eyeprint signature for the subject or group of subjects.

The retinal parameters used to generate the eyeprint signature include parameters that when combined, provide information sufficient to allow for the diagnosis of a neurodegenerative disease. The retinal parameters can be measured using art-recognized techniques that provide the desired information, and/or by using techniques or methods described herein. Examples of retinal parameters that can be measured include, but are not limited to, the thickness of the retinal nerve fiber layer, the amount of blood flowing in a selected retinal vessel, and the diameter of a selected retinal vessel. A variety of retinal parameters are known in the art. One of ordinary skill will recognize which retinal parameters can be used to generate an eyeprint signature. For example, the plurality of retinal parameters can be two, three, four, five or six different retinal parameters. The combination of the particular retinal parameters selected may vary depending on the disease or disorder being diagnosed. The parameters further may be selected based on a variety of other factors including, subjects being tested, progression of disease or disorder, etc. An eyeprint signature of the present invention can be used to diagnose and/or monitor the progression of a neurodegenerative disease.

In one embodiment, the selected retinal parameters include, but are not limited to, at least two of retinal nerve fiber layer thickness, retinal blood vessel diameter and retinal blood flow.

The term “subject” is known in the art, and, as used herein, refers to a warm-blooded animal, more preferably a mammal, including, e.g., non-human animals such as rats, mice, cats, dogs, sheep, horses, cattle, in addition to humans. In a preferred embodiment, the subject is a human. The subjects are those susceptible to developing a neurodegenerative disease.

In one aspect, optical coherence tomography measurements are used to measure the thickness of the retinal nerve fiber layer. As used herein the term, “optical coherence tomography” (OCT) refers to an imaging technique that produces high resolution cross sectional images of optical reflectivity. OCT systems use low-coherence interferometry to produce a two-dimensional image of optical scattering from internal tissue microstructures, i.e., distance information concerning various ocular structures is extracted from time delays of reflected signals. In OCT, light waves emitted by a superluminescent diode operating at 840 nm and between 200 μw and 1 mw are used to determine the images. The use of light waves enables OCT to achieve an axial resolution of 10 μm. Lateral resolution is approximately 70 μm.

OCT is particularly well suited for measuring retinal thickness due to this high level of resolution. A standard protocol measures the retinal nerve fiber layer thickness in a concentric peripapillary ring with the optic nerve head at the center

Measurements can be grouped into four quadrants in relation to the optic nerve head, i.e., temporal (towards the temple of the skull), superior (above the optic nerve head), nasal (towards the nose), and inferior (below the optic nerve head). In one aspect, measurements from a particular quadrant or quadrants of the retinal nerve fiber layer, i.e., temporal, superior, nasal, and/or inferior are used to generate an eyeprint signature.

One of skill in the art will recognize other techniques that can be used to measure the thickness of the retinal nerve fiber layer. For example, scanning laser polarimetry, and/or retinal nerve fiber layer photographs taken with a fundus camera can be used to measure the thickness of the retinal nerve fiber layer.

In another embodiment, retinal blood flow, and retinal blood vessel diameter measurements are obtained using a laser Doppler blood flowmeter, e.g., a Canon Laser Blood Flowmeter 100 (CLBF 100) (Canon, Tokyo, Japan). An advantage of this machine is that the measuring laser beam is locked onto the target blood vessel during eye movements through an eye-tracking feedback and control system. This feature is especially useful for measurements on patients suffering from Alzheimer's disease who are known to suffer from fixation instability. Feke G. T., et al, IEE Transact Biomed Engi (1987);34:673-680.

The laser Doppler blood flowmeter measures the blood vessel diameter (D), the centerline blood velocity (V), and the blood flow (F) in major retinal vessels. The measurement of the V is based on the principle of bidirectional laser Doppler velocimetry.

The laser Doppler blood flowmeter allows for measurements of blood velocity and blood flow in actual units of mm/s and μL/min respectively. These measurements allow for comparisons to be made within an eye and between the eyes of a subject, as well as between the eyes of one or more subjects.

As used herein the term “retinal blood vessel diameter” refers to the diameter of a blood vessel that supplies blood to the retina. In one embodiment, the retinal blood vessel selected is the major superior temporal retinal vein.

The term “retinal blood flow” refers to the amount of blood, measured in microliters per minute, which flows through the retinal blood vessel being measured.

In one embodiment, the present invention provides methods for diagnosing a neurodegenerative disease, e.g., Alzheimer's disease, based on a comparison of the subject's eyeprint signature to a standard eyeprint signature for the neurodegenerative disease. As used herein, the term “standard eyeprint signature for a neurodegenerative disease” refers to a plurality of retinal parameters, measured in a group of subjects previously diagnosed with the neurodegenerative disease, that are indicative of the presence of a neurodegenerative disease. The retinal parameters chosen indicate the current structural and functional characteristics of the retina in subjects known to be suffering from the neurodegenerative disease in question. For example, eyeprint signatures can be generated for a group of subjects who have all been previously diagnosed with a neurodegenerative disease. The same retinal parameters are measured in each subject. The values of the measurements of each retinal parameter, for all of the subjects, are then compiled, generating a standard eyeprint signature for the neurodegenerative disease.

Standard eyeprint signatures can also be generated at different stages of a neurodegenerative disease, e.g., a standard eyeprint signature can be generated for early stage Alzheimer's disease, middle stage Alzheimer's disease, and/or late stage Alzheimer's disease. For example, similar to the method described above, eyeprint signatures can be generated for a group of subjects previously diagnosed at a particular stage of a neurodegenerative disease. The values of the measurements of each retinal parameter, for all of the subjects, are then compiled, generating a standard eyeprint signature for the particular stage of the neurodegenerative disease.

Without wishing to be bound by any particular theory, it is believed that due to their differing pathology, different diseases will each generate a different standard eyeprint signature. Thus the standard eyeprint for one neurodegenerative disease, e.g., Alzheimer's disease, may differ from that of another neurodegenerative disease, e.g., multiple sclerosis.

In one embodiment, the present invention provides a method for monitoring the effectiveness of a therapeutic treatment for a neurodegenerative disease. For example, a monitoring eyeprint signature for a subject is generated and compared to a threshold eyeprint signature for the subject. The effectiveness of the treatment is then determined.

As used herein, the term “threshold eyeprint signature” refers to an eyeprint signature, as defined above, for a subject, or group of subjects, which is used as a baseline measurement for a comparison, i.e., examining or otherwise analyzing for similarities or differences, to the monitoring eyeprint. The threshold eyeprint signature can be generated at any time necessary including, prior to the subject starting a therapeutic treatment, during the course of a therapeutic treatment, or after a therapeutic treatment has concluded. Additionally, a threshold eyeprint signature can be generated at any stage of a neurodegenerative disease. For example, a threshold eyeprint signature can be generated during the early on-set stage of Alzheimer's disease, the middle stage of Alzheimer's disease, or the late stage of Alzheimer's disease.

The term “monitoring eyeprint signature” as used herein refers to an eyeprint signature, as defined above, for a subject or group of subjects, generated at a point in time subsequent to the generation of a threshold eyeprint signature for the same subject or group of subjects. For example, a threshold eyeprint signature can be generated for a subject. At a desired time thereafter, a monitoring eyeprint signature is generated. The selected retinal parameters used to generate the monitoring eyeprint signature are the same as the retinal parameters used to generate the threshold eyeprint signature to which the monitoring eyeprint signature will be compared.

In one embodiment, the comparison between the threshold eyeprint signature and the monitoring eyeprint signature is made in order to evaluate the effectiveness of a therapeutic treatment. The therapeutic treatment may have been begun prior to generation of the threshold eyeprint signature, or after the threshold eyeprint signature has been generated but must be started before the monitoring eyeprint signature is generated.

What indicates an effective therapeutic treatment will depend on the neurodegenerative disease being evaluated, and the selected retinal parameters. For example, if the therapeutic treatment is being evaluated for it's effectiveness in treating Alzheimer's disease, and the selected retinal parameters are the thickness of the retinal nerve fiber layer, the blood flow through a selected retinal blood vessel, and the diameter of the selected retinal blood vessel, a monitoring eyeprint signature that reveals a decrease in the values of the selected retinal parameters based on a comparison to the values of the selected retinal parameters comprising the threshold eyeprint signature for the subject indicates an ineffective treatment.

In another embodiment, the present invention provides a method for monitoring the progression of a neurodegenerative disease. In one aspect, this is achieved by generating a threshold eyeprint signature for a subject. A monitoring eyeprint signature for a subject is subsequently generated. The monitoring eyeprint signature is then compared to the threshold eyeprint signature for the subject, and the progression of the neurodegenerative disease is determined.

What indicates a progression of the neurodegenerative disease will depend on the neurodegenerative disease being evaluated, as well as the selected retinal parameters. For example, if the neurodegenerative disease being evaluated is Alzheimer's disease, and the selected retinal parameters are the thickness of the retinal nerve fiber layer, the blood flow through a selected retinal blood vessel, and the diameter of the selected retinal blood vessel, then a monitoring eyeprint signature that reveals a decrease in the values of the selected retinal parameters based on a comparison to the values of the selected retinal parameters comprising the threshold eyeprint signature for the subject indicates a progression of Alzheimer's disease.

In another embodiment, a control eyeprint signature is generated. As used herein a “control eyeprint signature” refers to an eyeprint signature, as defined above, for a subject or group of subjects who have had a negative diagnosis for the neurodegenerative disease being evaluated. For example, a subject or group of subjects are selected who have had a negative diagnosis for the neurodegenerative disease in question. A plurality of retinal parameters are measured in the subject or subjects. The retinal parameters selected for generating the control eyeprint signature are the same as the retinal parameters used to generate the eyeprint signature to which the control eyeprint signature is being compared. For example, if the control eyeprint signature is being compared to a standard eyeprint signature for a neurodegenerative disease generated based on the measurements of the thickness of the retinal nerve fiber layer, the blood flow through a selected retinal blood vessel, and the diameter of the selected retinal blood vessel, those same retinal parameters would be used to generate the control eyeprint signature.

### EXAMPLES

The following materials, methods and examples are meant to be illustrative only and are not intended to be limiting.

### Generation of Standard Eyeprint for a Neurodegenerative Disease—

a) Alzheimer's Disease

Eleven patients (mean age 77±5 years) with a diagnosis of Alzheimer's disease and mild dementia based on the Mini Mental State Examination (MMSE) (mean score 24.5 out of a maximum of 30) were selected. Retinal nerve fiber layer thickness, retinal blood flow and retinal blood vessel diameter were the selected retinal parameters used to generate the standard eyeprint signature.

Retinal nerve fiber layer thickness was measured using OCT. Average and segmental (four quadrants) retinal nerve fiber layer thickness values were measured.

A retinal laser Doppler blood flow instrument was used to measure the retinal blood flow and blood vessel diameter of the major superior temporal retinal vein.

Table 1 shows the results of these tests.

TABLE 1

| | Average | Average | Average | |
|---|---|---|---|---|
| | Overall | Retinal Nerve | Retinal | Average |
| | Retinal | Fiber Layer | Blood | Retinal |
| | Nerve | Thickness of | Flow | Blood |
| | Fiber Layer | the Superior | (microliters | Vessel |
| | Thickness | Quadrant | per | Diameter |
| | (microns) | (microns) | minute) | (microns) |
| Subjects Pre- | 81.1 ± 13.0 | 87.4 ± 17.5 | 11.9 ± 4.4 | 133 ± 10 |
| diagnosed with | | | | |
| Alzheimer's | | | | |
| disease | | | | |
| (n = 11) | | | | |

The results generated are used as a standard eyeprint signature for diagnosing Alzheimer's disease.

b) Inflammatory Optic Neuropathy

Twelve patients (mean age 62±11 years) with a diagnosis of inflammatory optic neuropathy were selected. Retinal nerve fiber layer thickness, retinal blood flow, and retinal blood vessel diameter were the selected retinal parameters used to generate the standard eyeprint signature.

Retinal nerve fiber layer thickness was measured using OCT. Average and segmental (four quadrants) retinal nerve fiber layer thickness values were measured.

A retinal laser Doppler blood flow instrument was used to measure the retinal blood flow and blood vessel diameter of the major superior temporal retinal vein.

Table 2 shows the results of these tests.

TABLE 2

| | | Average | Average | | |
|---|---|---|---|---|---|
| | | Retinal | Retinal | | |
| | Average | Never Fiber | Nerve Fiber | | Average |
| | Overall | Layer | Layer | Average | Retinal |
| | Retinal Nerve | Thickness of | Thickness of | Retinal | Blood |
| | Fiber Layer | the Nasal | the Superior | Blood Flow | Vessel |
| | Thickness | Quadrant | Quadrant | (microliters | Diameter |
| | (microns) | (microns) | (microns) | per minute) | (microns) |
| Subjects Pre- | 83.9 ± 7.5 | 54.1 ± 11.7 | 85.3 ± 15.1 | 17.5 ± 5.1 | 143 ± 10 |
| diagnosed with | | | | | |
| inflammatory | | | | | |
| optic | | | | | |
| neuropathy | | | | | |
| (n = 12) | | | | | |

The results generated are used as a standard eyeprint signature for diagnosing inflammatory optic neuropathy.

c) Glaucoma

Thirteen patients (mean age 72±7 years) with a diagnosis of glaucoma were selected. Retinal nerve fiber layer thickness, retinal blood flow and retinal blood vessel diameter were the selected retinal parameters used to generate the standard eyeprint signature.

Retinal nerve fiber layer thickness was measured using OCT. Average and segmental (four quadrants) retinal nerve fiber layer thickness values were measured.

A retinal laser Doppler blood flow instrument was used to measure the retinal blood flow and blood vessel diameter of the major superior temporal retinal vein.

Table 3 shows the results of these tests.

TABLE 3

| | | Average | | |
|---|---|---|---|---|
| | | Retinal | | |
| | Average | Nerve Fiber | | Average |
| | Overall | Layer | Average | Retinal |
| | Retinal Nerve | Thickness of | Retinal | Blood |
| | Fiber Layer | the Inferior | Blood Flow | Vessel |
| | Thickness | Quadrant | (microliters | Diameter |
| | (microns) | (microns) | per minute) | (microns) |
| Subjects Pre- | 81.5 ± 6.2 | 86.4 ± 12.4 | 8.8 ± 3.6 | 131 ± 10 |
| diagnosed with | | | | |
| glaucoma | | | | |
| (n = 13) | | | | |

The results generated are used as a standard eyeprint signature for diagnosing inflammatory optic neuropathy.

### Diagnosis of a Neurodegenerative Disease Using a Standard Eyeprint Signature for the Disease.

a) Alzheimer's Disease

An eyeprint signature for a subject is generated. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the eyeprint signature of the subject.

A comparison is then made between the subject's eyeprint signature and the standard eyeprint signature for Alzheimer's disease. The subject may be afflicted with Alzheimer's disease if their eyeprint signature corresponds to the standard eyeprint signature for Alzheimer's disease. Further testing to confirm may be required.

### Monitoring the Effectiveness of a Therapeutic Treatment for a Neurodezenerative Disease.

a) Monitoring the Effectiveness of a Therapeutic Treatment for Alzheimer 's Disease.

A threshold eyeprint signature is generated for a subject previously diagnosed with Alzheimer's disease. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the threshold eyeprint signature of the subject.

The therapeutic treatment for Alzheimer's disease may start after the threshold eyeprint is generated, or it may have already been started.

After the threshold eyeprint signature has been generated, and the therapeutic treatment has begun, a monitoring eyeprint signature for the subject is generated. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the monitoring eyeprint signature of the subject.

A comparison is then made between the monitoring eyeprint signature and the threshold eyeprint signature. A decrease in the values of the selected retinal parameters comprising the monitoring eyeprint as compared to the values of the threshold eyeprint signature indicates an ineffective treatment.

b) Monitoring the Effectiveness of a Therapeutic Treatment for Inflammatory Optic Neuropathy.

A threshold eyeprint signature is generated for a subject previously diagnosed with inflammatory optic neuropathy. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the threshold eyeprint signature of the subject.

The therapeutic treatment for inflammatory optic neuropathy may start after the threshold eyeprint is generated, or it may have already been started.

After the threshold eyeprint signature has been generated and after the therapeutic treatment has begun, a monitoring eyeprint signature for the subject is generated. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the monitoring eyeprint signature of the subject.

A comparison is then made between the monitoring eyeprint signature and the threshold eyeprint signature. A monitoring eyeprint that shows a decrease in the thickness of the retinal nerve fiber layer in the superior and nasal quadrants indicates an ineffective treatment.

### Monitoring the Progression of a Neurodegenerative Disease.

a) Monitoring the Progression of Alzheimer's Disease

A threshold eyeprint signature is generated for a subject previously diagnosed with Alzheimer's disease. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the threshold eyeprint signature of the subject.

At a subsequent point in time, a monitoring eyeprint signature for the subject is generated. More specifically, retinal nerve fiber layer thickness, retinal blood flow and retinal blood vessel diameter are selected as retinal parameters. Retinal nerve fiber layer thickness is measured using OCT. Average and segmental (four quadrants) retinal nerve fiber layer thickness values are measured.

A retinal laser Doppler blood flow instrument is used to measure the retinal blood flow and blood vessel diameter of the major superior temporal retinal vein. The results generated comprise the monitoring eyeprint signature of the subject.

A comparison is then made between the monitoring eyeprint signature and the threshold eyeprint signature. A decrease in the values of the selected retinal parameters comprising the monitoring eyeprint as compared to the values of the threshold eyeprint signature indicates the progression of Alzheimer's disease in the subject.

### Other Embodiments

It is to be understood that while the invention has been described in conjunction with the detailed description thereof, the foregoing description is intended to illustrate, and not limit the scope of the invention, which is defined by the scope of the appended claims. Other aspects, advantages, and modifications are within the scope of the following claims.

In addition, the contents of all patent publications discussed supra are incorporated in their entirety by this reference.

It is to be understood that wherever values and ranges are provided herein, e.g., of nerve fiber layer thickness, retinal blood flow, and retinal blood vessel diameters, all values and ranges encompassed by these values and ranges, are meant to be encompassed within the scope of the present invention. Moreover, all values that fall within these ranges, as well as the upper or lower limits of a range of values, are also contemplated by the present application.

1. A method for diagnosing a neurodegenerative disease in a subject, comprising:
generating an eyeprint signature for a subject based on measurements of a plurality of selected retinal parameters;
and diagnosing whether the subject has the neurodegenerative disease based on a comparison between the eyeprint signature of the subject and a standard eyeprint signature for the neurodegenerative disease.

2. The method of claim 1, wherein the selected retinal parameters are at least two parameters selected from the group consisting of the thickness of a retinal nerve fiber layer, the diameter of a retinal blood vessel, and a retinal blood flow rate.

3. The method of claim 2, wherein the thickness of the retinal nerve fiber layer is measured using an optical coherence tomography machine.

4. The method of claim 3, wherein the thickness of the superior, temporal, inferior, and/or nasal quadrants of the retinal nerve fiber layer are measured.

5. The method of claim 2, wherein the diameter of the retinal blood vessel and/or the retinal blood flow rate areas measured using a laser Doppler blood flowmeter.

6. (canceled)

7. The method of claim 1, wherein the neurodegenerative disease is a neurodegenerative disease of the eye.

8. The method of claim 1, wherein the neurodegenerative disease is selected from the group consisting of inflammatory optic neuropathy, macular degeneration, glaucoma, retinitis pigmentosa, and diabetic retinopathy.

9. The method of claim 1, wherein the neurodegenerative disease is selected from the group consisting of Alzheimer's disease, Parkinson's disease, multiple sclerosis, amyotrophic lateral sclerosis, and Huntington's disease.

10. A method for diagnosing a neurodegenerative disease in a subject according to claim 1, wherein the neurodegenerative disease is Alzheimer's disease, comprising:
generating an eyeprint signature for a subject based on measurements of a plurality of selected retinal parameters;
and diagnosing whether the subject has Alzheimer's disease based on a comparison between the eyeprint signature of the subject and a standard eyeprint signature for Alzheimer's disease.

11. The method of claim 10, wherein the selected retinal parameters are at least two parameters selected from the group consisting of the thickness of a retinal nerve fiber layer, the diameter of a retinal blood vessel, and a retinal blood flow rate.

12.-15. (canceled)

16. The method of claim 11, wherein the standard eyeprint signature for Alzheimer's disease comprises:
a decreased thickness of the retinal nerve fiber layer;
a decreased diameter of the retinal blood vessel;
and a decreased blood flow rate, based on a comparison to a control eyeprint signature.

17. (canceled)

18. The method of claim 11, wherein the standard eyeprint signature for Alzheimer's disease comprises:
a superior quadrant retinal nerve fiber layer thickness in the range from about 70 microns to about 105 microns;
a retinal blood vessel diameter in the range from about 122 microns to about 142 microns;
and a retinal blood flow in the range from about 8 μL/min to about 18 μL/min.

19. A method for diagnosing a neurodegenerative disease in a subject according to claim 1, wherein the neurodegenerative disease is Parkinson's disease, comprising:
generating an eyeprint signature for a subject based on measurements of a plurality of selected retinal parameters;
and diagnosing whether the subject has Parkinson's disease based on a comparison between the eyeprint signature of the subject and a standard eyeprint signature for Parkinson's disease.

20. The method of claim 19, wherein the selected retinal parameters are at least two parameters selected from the group consisting of the thickness of a retinal nerve fiber layer, the diameter of a retinal blood vessel, and a retinal blood flow rate.

21.-24. (canceled)

25. A method for diagnosing a neurodegenerative disease in a subject according to claim 1, wherein the neurodegenerative disease is glaucoma, comprising:
generating an eyeprint signature for a subject based on measurements of a plurality of selected retinal parameters;
and diagnosing whether the subject has glaucoma based on a comparison between the eyeprint signature of the subject and a standard eyeprint signature for glaucoma.

26. The method of claim 25, wherein the selected retinal parameters are at least two parameters selected from the group consisting of the thickness of a retinal nerve fiber layer, the diameter of a retinal blood vessel, and a retinal blood flow rate.

27.-30. (canceled)

31. The method of claim 26, wherein the standard eyeprint signature for glaucoma comprises:
a decreased thickness of the retinal nerve fiber layer;
a decreased diameter of the retinal blood vessel;
and a decreased blood flow rate, based on a comparison to a control eyeprint signature.

32. (canceled)

33. The method of claim 26, wherein a standard eyeprint signature for glaucoma comprises:
an inferior quadrant retinal nerve fiber layer thickness in the range from about 74 microns to about 99 microns;
a retinal blood vessel diameter in the range from about 88 microns to about 123 microns;
and a retinal blood flow in the range from about 5 μL/min to about 13 μL/min.

34. A method for diagnosing a neurodegenerative disease in a subject according to claim 1, wherein the neurodegenerative disease is inflammatory optic neuropathy, comprising:
generating an eyeprint signature for a subject based on measurements of a plurality of selected retinal parameters;
and diagnosing whether the subject has inflammatory optic neuropathy based on a comparison between the eyeprint signature of the subject and a standard eyeprint signature for inflammatory optic neuropathy.

35. The method of claim 34, wherein the selected retinal parameters are at least two parameters selected from the group consisting of the thickness of a retinal nerve fiber layer, the diameter of a retinal blood vessel, and a retinal blood flow rate.

36.-39. (canceled)

40. The method of claim 35, wherein the standard eyeprint signature for inflammatory optic neuropathy comprises:
a decreased thickness of the retinal nerve fiber layer;
a normal diameter of the retinal blood vessel;
and a normal blood flow rate, based on a comparison to a control eye print signature.

41. (canceled)

42. The method of claim 35, wherein a standard eyeprint signature for inflammatory optic neuropathy comprises:
a superior quadrant retinal nerve fiber layer thickness in the range from about 70 microns to about 100 microns;
an inferior quadrant retinal nerve fiber layer thickness in the range from about 101 to about 130;
a retinal blood vessel diameter in the range from about 133 microns to about 153 microns;
and a retinal blood flow in the range from about 12 μL/min to about 23 μL/min.

43. A method for monitoring a therapeutic treatment for a neurodegenerative disease, comprising:
generating a monitoring eyeprint signature for a subject based on a plurality of selected retinal parameters; comparing the monitoring eyeprint signature to a threshold eyeprint signature for the subject;
and determining the effectiveness of the therapeutic treatment.

44. The method of claim 43, wherein the selected retinal parameters are at least two parameters selected from the group consisting of the thickness of a retinal nerve fiber layer, the diameter of a retinal blood vessel, and a retinal blood flow rate.

45. The method of claim 44, wherein the thickness of the retinal nerve fiber layer is measured using an optical coherence tomography machine.

46. The method of claim 44, wherein the thickness of the superior, temporal, inferior, and/or nasal quadrants of the retinal nerve fiber layer are measured.

47. The method of claim 44, wherein the diameter of the retinal blood vessel and/or the retinal blood flow rate are measured using a laser Doppler blood flowmeter.

48. (canceled)

49. The method of claim 43, wherein the neurodegenerative disease is a neurodegenerative disease of the eye.

50. The method of claim 43, wherein the neurodegenerative disease is selected from the group consisting of optic neuropathy, macular degeneration, glaucoma, retinitis pigmentosa, and diabetic retinopathy.

51. The method of claim 43, wherein the neurodegenerative disease is selected from the group consisting of Alzheimer's disease, Parkinson's disease, multiple sclerosis, amyotrophic lateral sclerosis, and Huntington's disease.

52. The method of claim 43, wherein the neurodegenerative disease is Alzheimer's disease.

53. The method of claim 52, wherein the selected retinal parameters comprising the monitoring eyeprint are at least two parameters selected from the group consisting of the thickness of a retinal nerve fiber layer, the diameter of a retinal blood vessel, and the retinal blood flow rate.

54.-57. (canceled)

58. The method of claim 53, wherein a decrease in the values of the selected retinal parameters comprising the monitoring eyeprint based on a comparison to the threshold eyeprint indicates an ineffective treatment.

59. The method of claim 43, wherein the neurodegenerative disease is Parkinson's disease.

60. The method of claim 59, wherein the selected retinal parameters comprising the monitoring eyeprint are at least the retinal nerve fiber layer thickness, retinal blood vessel diameter, and retinal blood flow.

61.-64. (canceled)

65. The method of claim 43, wherein the neurodegenerative disease is glaucoma.

66. The method of claim 65, wherein the selected retinal parameters comprising the monitoring eyeprint are at least the retinal nerve fiber layer thickness, the retinal blood vessel diameter, and the retinal blood flow.

67.-70. (canceled)

71. The method of claim 66, wherein a decrease in the values of the selected retinal parameters comprising the monitoring eyeprint based on a comparison to the threshold eyeprint indicates an ineffective treatment.

72. The method of claim 43, wherein the neurodegenerative disease is inflammatory optic neuropathy.

73. The method of claim 72, wherein the selected retinal parameters comprising the monitoring eyeprint are at least the retinal nerve fiber layer thickness, retinal blood vessel diameter, and retinal blood flow.

74.-77. (canceled)

78. The method of claim 73, wherein a decrease in the thickness of the retinal nerve fiber layer and an increase in the blood flow rate measurements comprising the monitoring eyeprint based on a comparison to the threshold eyeprint indicates an ineffective treatment.

79. A method for monitoring the progression of a neurodegenerative disease, comprising:
generating a monitoring eyeprint signature for a subject based on a plurality of selected retinal parameters;
comparing the monitoring eyeprint signature to a threshold eyeprint signature for the subject;
and determining the progression of the neurodegenerative disease.

80. The method of claim 79, wherein the selected retinal parameters are at least two parameters selected from the group consisting of the thickness of a retinal nerve fiber layer, the diameter of a retinal blood vessel, and a retinal blood flow rate.

81. The method of claim 80, wherein the thickness of the retinal nerve fiber layer is measured using an optical coherence tomography machine.

82. The method of claim 80, wherein the thickness of the superior, temporal, inferior, and/or nasal quadrants of the retinal nerve fiber layer are measured.

83. The method of claim 80, wherein the diameter of the retinal blood vessel and/or the retinal blood flow rate are measured using a laser Doppler blood flowmeter.

84. (canceled)

85. The method of claim 79, wherein the neurodegenerative disease is a neurodegenerative disease of the eye.

86. The method of claim 79, wherein the neurodegenerative disease is selected from the group consisting of inflammatory optic neuropathy, macular degeneration, glaucoma, retinitis pigmentosa, and diabetic retinopathy.

87. The method of claim 79, wherein the neurodegenerative disease is selected from the group consisting of Alzheimer's disease, Parkinson's disease, multiple sclerosis, amyotrophic lateral sclerosis, and Huntington's disease.

88. The method of claim 79, wherein the neurodegenerative disease is Alzheimer's disease.

89. The method of claim 88, wherein the selected retinal parameters comprising the monitoring eyeprint are at least the retinal nerve fiber layer thickness, the retinal blood vessel diameter, and the retinal blood flow.

90.-93. (canceled)

94. The method of claim 89, wherein a decrease in the values of the selected retinal parameters comprising the monitoring eyeprint based on a comparison to the threshold eyeprint indicates the progression of Alzheimer's disease.

95. The method of claim 79, wherein the neurodegenerative disease is Parkinson's disease.

96. The method of claim 95, wherein the selected retinal parameters comprising the monitoring eyeprint are at least the retinal nerve fiber layer thickness, retinal blood vessel diameter, and retinal blood flow.

97.-100. (canceled)

101. The method of claim 79, wherein the neurodegenerative disease is glaucoma.

102. The method of claim 101, wherein the selected retinal parameters comprising the monitoring eyeprint are at least the retinal nerve fiber layer thickness, the retinal blood vessel diameter, and the retinal blood flow.

103.-106. (canceled)

107. The method of claim 102, wherein a decrease in the values of the selected retinal parameters comprising the monitoring eyeprint based on a comparison to the threshold eyeprint indicates an ineffective treatment.

108. The method of claim 79, wherein the neurodegenerative disease is inflammatory optic neuropathy.

109. The method of claim 108, wherein the selected retinal parameters comprising the monitoring eyeprint are at least the retinal nerve fiber layer thickness, retinal blood vessel diameter, and retinal blood flow.

110.-113. (canceled)

114. The method of claim 109, wherein a decrease in the thickness of the retinal nerve fiber layer measurement comprising the monitoring eyeprint based on a comparison to the threshold eyeprint indicates an ineffective treatment.

115. A method of screening a subject for a neurodegenerative disease comprising:
comparing an eyeprint signature for a subject to a standard eyeprint signature for the neurodegenerative disease, wherein a correlation between the eyeprint signature for the subject and the standard eyeprint signature for the neurodegenerative disease indicates the presence of a neurodegenerative disease such that the subject is screened.

116. The method of claim 115, wherein the neurodegenerative disease is a neurodegenerative disease of the eye.

117. The method of claim 115, wherein the neurodegenerative disease is selected from the group consisting of inflammatory optic neuropathy, macular degeneration, glaucoma, retinitis pigmentosa, and diabetic retinopathy.

118. The method of claim 115, wherein the neurodegenerative disease is selected from the group consisting of Alzheimer's disease, Parkinson's disease, multiple sclerosis, amyotrophic lateral sclerosis, and Huntington's disease.

119. The method of claim 115, wherein the neurodegenerative disease is Alzheimer's disease.

120. The method of claim 119, wherein the standard eyeprint signature for Alzheimer's disease comprises:
a superior quadrant retinal nerve fiber layer thickness in the range from about 70 microns to about 105 microns;
a retinal blood vessel diameter in the range from about 122 microns to about 142 microns;
and a retinal blood flow in the range from about 8 μL/min to about 18 μL/min.

121. The method of claim 115, wherein the neurodegenerative disease is Parkinson's disease.

122. The method of claim 115, wherein the neurodegenerative disease is glaucoma.

123. The method of claim 122, wherein the standard eyeprint signature for glaucoma comprises:
an inferior quadrant retinal nerve fiber layer thickness in the range from about 74 microns to about 99 microns;
a retinal blood vessel diameter in the range from about 88 microns to about 123 microns;
and a retinal blood flow in the range from about 5 μL/min to about 13 μL/min.

124. The method of claim 115, wherein the neurodegenerative disease is inflammatory optic neuropathy.

125. The method of claim 124, wherein the standard eyeprint signature for inflammatory optic neuropathy comprises:
a superior retinal nerve fiber layer thickness in the range from about 70 microns to about 100 microns;
an inferior quadrant retinal nerve fiber layer thickness in the range from about 101 microns to about 130 microns;
a retinal blood vessel diameter in the range from about 133 microns to about 153 microns;
and a retinal blood flow in the range from about 12 μL/min to about 23 μL/min.
